(19) United States
(12) Patent Application Publication
(10) Pub. No.: US 2010/0081981 A1
(43) Pub. Date: Apr. 1, 2010

(54) WRIST SUPPORT

(76) Inventor: Shien-Che CHENG, Taipei City (TW)

Correspondence Address: RABIN & Berdo, PC, 1101 14TH STREET, NW, SUITE 500, WASHINGTON, DC, 20005, US

(21) Appl. No.: 12/243,532

(22) Filed: Oct. 1, 2008

(51) Int. Cl. A61F 5/01

(52) U.S. Cl. 602/21

(57) ABSTRACT

A wrist support is described, which is suitable for assisting a user to restrain the motion and flexion of a wrist and protect the wrist. The wrist support includes a planar assembly, a finger-wearing set, at least one accommodating pocket, a first tightening strip set, a second tightening strip set, and a plurality of supporting members. The wrist support wraps a hand back, a first metacarpal bone outer side of the hand, a fifth metacarpal bone outer side of the hand, a wrist, and a portion of a forearm, and uses the supporting members to provide a protection function and suppress the flexion of the wrist, so as to maintain the gesture. The supporting members may be accommodated in the accommodating pockets at will according to users' personal requirements. The wrist support can be worn and used either on a left hand or on a right hand.

### BACKGROUND OF THE INVENTION

1. Field of the Invention

The present invention relates to a wrist support, and more particularly to a wrist support, which can assist a user to restrain the motion and flexion of the wrist and maintain a gesture when doing sports, so as to protect the wrist. The wrist support can be worn and used either on a left hand or on a right hand, and meanwhile can be used as a protector for holding the wrist in a correction therapy.

2. Related Art

Generally speaking, various sports, such as golf and bowling, require controlling the flexion of the wrist, which also needs to protect the wrist from being injured. When playing golf, the flexion of the wrist that leads the club needs to be controlled properly when swinging the club. If the wrist is improperly and slightly flexed, the impact face of the club head is not correct, which results in the hook and slice, and the distance of the shot is also affected. As for bowling, the flexion of the wrist should also be paid more attention to, so as to ensure a successful shot, and meanwhile the wrist shall be protected from being injured.

Therefore, many gloves or auxiliary equipments for correcting a motion of the wrist have been put forward or proposed on the market. These equipments all provide auxiliary functions to enable the user to restrain the motion and flexion of the wrist when doing sports. Although the above auxiliary correcting devices can solve some of the flexion problems of the hand and wrist, most fixed wrist supports merely focus on the hand back portion, so that the auxiliary suppressing effect is fairly limited. Moreover, the existing auxiliary correcting devices can only be worn on one of the left hand and the right hand, and cannot be used by both the left hand and the right hand.

Taiwan Patent No. 336501 has disclosed a thumb through hole, which easily slips, becomes unstable, has only one supporting member, cannot be alternatively used by both the left hand and the right hand. Golf gloves disclosed in US Patent No. 2006/281753, U.S. Pat. No. 6,539,551, and U.S. Pat. No. 5,638,548 are merely suitable for being used in the golf sports, which cannot restrain the hand and wrist from bending towards the direction of the first metacarpal bone side or the fifth metacarpal bone side by the supporting members thereof and cannot protect the whole wrist. In addition, the supporting members cannot be replaced, which thus cannot provide proper forces to different users to cater to individuals' comfort levels and may bring inconveniences to the users. The gloves disclosed above cannot be alternatively used by both the left hand and the right hand, and cannot be used as wrist braces for correction therapy as well.

For example, wrist braces dedicated to bowling disclosed in U.S. Pat. No. 5,839,124, U.S. Pat. No. 5,708,981, U.S. Pat. No. 5,445,566, U.S. Pat. No. 5,427,577, U.S. Pat. No. 4,198,709, U.S. Pat. No. 4,190,906, and U.S. Pat. No. 4,138,108 are designed to respectively support the palm back and the palm center or only support the palm back, and some of them are designed with a thumb wearing hole. However, none of the above disclosed wrist braces can restrain the hand and wrist from bending towards the direction of the first metacarpal bone side or the fifth metacarpal bone side and protect the whole wrist at the same time. In addition, there are still many other wrist braces dedicated to bowling, which are rather complex in design and high in cost.

For example, U.S. Pat. No. 6,199,208 has disclosed an athletic wrist support, which easily slips and cannot restrain the hand and wrist from bending towards the direction of the first metacarpal bone side or the fifth metacarpal bone side. U.S. Pat. No. 5,933,868 has disclosed a sports glove, which only suppresses the palm back.

For example, US2007225630, US2006149180, US2005197609, U.S. Pat. No. 7,318,812, U.S. Pat. No. 6,893,410, U.S. Pat. No. 6,341,376, and U.S. Pat. No. 5,873,130 have disclosed many wrist devices for protection, correction, and rehabilitation, most of which only have functions of fixing the palm and the wrist and maintaining the gesture, cannot be applied to various sports.

Although wrist braces disclosed in US2007225630 and U.S. Pat. No. 7,318,812 are suitable for being worn on both the left hand and the right hand, the fastening straps thereof have complex structures and are thus inconvenient for wearing and using. In addition, U.S. Pat. No. 5,267,943 has disclosed a wrist and a hand support device, which are designed for workers responsible for heavy work such as construction workers, cannot restrain the hand and wrist from bending towards the direction of the first metacarpal bone side or the fifth metacarpal bone side, and cannot be alternatively used by both the left hand and the right hand as well.

### SUMMARY OF THE INVENTION

Accordingly, the present invention is directed to a wrist support, which can solve problems of the prior art that a deviation or a fault occurs when doing sports since a glove or a protector in the prior art cannot effectively maintain a wrist of a user in a fixed gesture, supporting members in the glove or protector in the prior art cannot be replaced at will according to the user's requirements, and the glove or protector in the prior art is not suitable for being worn on a left hand and on a right hand at the same time.

The wrist support provided in the present invention is used to wrap a hand back, a first metacarpal bone outer side of the hand, a fifth metacarpal bone outer side of the hand (a hand knife side), a wrist, and a portion of a forearm of a user, so as to assist the user to suppress flexion of the wrist, maintain a fixed gesture, and protect the wrist when doing sports. Besides being used when doing sports, the wrist support is further used as a protector for holding the wrist in a correction therapy.

The wrist support of the present invention includes a planar assembly, a finger-wearing set, at least one accommodating pocket, at least one supporting member, a first tightening strip set, and a second tightening strip set. The planar assembly includes a hand back portion, a thumb side portion, a hand knife portion, and a wrist portion, so as to wrap the hand back, the first metacarpal bone outer side of the hand, the fifth metacarpal bone outer side of the hand (the hand knife side), the wrist, and a forepart of the arm of the user. The planar assembly is also provided with a thumb fitting hole for the thumb to penetrate there through. The finger-wearing set is disposed on one side of the hand back portion of the planar assembly corresponding to the other fingers except the thumb, and is formed with at least one finger hole for the other fingers except the thumb to penetrate there through. The accommodating pockets longitudinally correspond to a longitudinal connecting line between the hand, the wrist and the arm, and are evenly disposed in the planar assembly in a parallel and adjacent manner. Each accommodating pocket has an accommodating space therein and is provided with an opening and a cover on the other end thereof opposite to the finger-wearing set. The supporting members can be selectively mounted in the accommodating pocket at any moment, so as to offer the effective stability and rational support to the user's hand, wrist and forearm and protect them from being impacted and crashed by a foreign object.

The first tightening strip set includes a first tightening strip and a first Velcro portion. One end of the first tightening strip is fixed at an edge position of the wrist portion of the planar assembly. The first Velcro portion is mounted on the other end of the first tightening strip. The second tightening strip set includes a second tightening strip and a second Velcro portion. One end of the second tightening strip is fixed at an edge position of the thumb side portion of the planar assembly. The second Velcro portion is mounted on the other end of the second tightening strip. The two surfaces of the hand back portion of the planar assembly are respectively provided with adhering strips having loop structures. Hook structures of the two Velcro portions are tightly adhered to the loop structures of the adhering strips, such that the two tightening strip sets are adhered to the hand back portion of the planar assembly, thereby tightening to the arm to fix the wrist support. In addition, the Velcro portions of the two tightening strip sets can be easily detached from the tightening strips to change adhering directions thereof, such that the wrist support is inverted, which thus is suitable for being worn on both the left hand and the right hand.

The efficacy of the present invention lies in that, the supporting members mounted in the accommodating pocket are used to restrain the motion and flexion of the hand and the wrist towards the palm back, palm center, the first metacarpal bone side or the fifth metacarpal bone side, thereby effectively and stably maintaining the gesture of the hand and the wrist, and protecting the hand and the wrist. The wrist support can also help some beginners of sports, who need to pay more attention to the gesture of the wrist, to understand the correct key principles and become accustomed to using the wrist more easily, and meanwhile allows the user to move his/her fingers freely and flexibly. In addition, since the Velcro portions of the tightening strip sets are easily detached to change the directions in the present invention, the wrist support is not restricted to being worn on the left hand or the right hand, thereby achieving great economic benefits both in terms of manufacturing cost and users' consumption.

As for the supporting members of the present invention, the type of the supporting members can be replaced, and the number of the supporting members can be increased, or reduced at will and at any moment according to the user's actual requirements, thereby achieving the highest comfort degree and correcting an undesirable flexion of the wrist. The present invention is further characterized in that, when the wrist is injured, the wrist support is further used as a fixed rigid wrist brace for corrective rehabilitation to completely hold the wrist joint, thereby protecting the wrist from being injured once again. In this case, the wrist support achieves the functions and benefits of a rigid wrist brace. Meanwhile, the fingers can be moved freely and flexibly, so as to promote blood circulation and enhance the rehabilitation and correction efficacies. If the wrist is injured, after the fixed rigid wrist brace is worn for a period of time, the number of the used supporting members may be gradually reduced to enable the wrist to flex to a certain extent, thereby promoting the blood circulation and enhancing the rehabilitation effect. Furthermore, the supporting members may be totally taken out, so that the wrist support achieves the benefits of a soft wrist brace.

### BRIEF DESCRIPTION OF THE DRAWINGS

The present invention will become more fully understood from the detailed description given herein below for illustration only, and thus is not limitative of the present invention, and wherein:

FIG. 1 is an exploded view of a wrist support of the present invention;

FIG. 2 is a schematic view of the wrist support of the present invention;

FIG. 3A is a schematic view of motions of mounting a supporting member into an accommodating pocket according to the present invention;

FIG. 3B is a schematic view of motions of mounting the supporting member into the accommodating pocket according to the present invention;

FIG. 3C is a schematic view of motions of mounting the supporting member into the accommodating pocket according to the present invention;

FIG. 4A is a schematic view of a wrist support worn on a left hand according to the present invention;

FIG. 4B is a schematic view of the wrist support worn on a left hand according to the present invention;

FIG. 4C is a schematic front view of the wrist support worn on a hand of a user according to the present invention;

FIG. 4D is a schematic view of the wrist support worn on a right hand according to the present invention;

FIG. 4E is a schematic view of the wrist support worn on a right hand according to the present invention;

FIG. 5A is a schematic view of an elastic band of the wrist support in forming a different number of finger holes according to the present invention;

FIG. 5B is a schematic view of the elastic band of the wrist support in forming a different number of finger holes according to the present invention;

FIG. 5C is a schematic view of the elastic band of the wrist support in forming a different number of finger holes according to the present invention;

FIG. 5D is a schematic view of the elastic band of the wrist support in forming a different number of finger holes according to the present invention;

FIG. 5E is a schematic view of the elastic band of the wrist support in forming a different number of finger holes according to the present invention;

FIG. 6A is an exploded view of a combination of a tightening strip and a Velcro portion in a different configuration according to the present invention;

FIG. 6B is a schematic assembled view of a combination of the tightening strip and the Velcro portion in a different configuration according to the present invention;

FIG. 6C is a schematic assembled view of a combination of the tightening strip and the Velcro portion in a different configuration according to the present invention;

FIG. 7A is a schematic exploded view of a combination of a tightening strip and a Velcro portion in a different configuration according to the present invention; and

FIG. 7B is a schematic assembled view of a combination of the tightening strip and the Velcro portion in a different configuration according to the present invention.

### DETAILED DESCRIPTION OF THE INVENTION

To make the objectives, structures, features, and functions of the present invention more comprehensible, the present invention is illustrated below in detail through the embodiments.

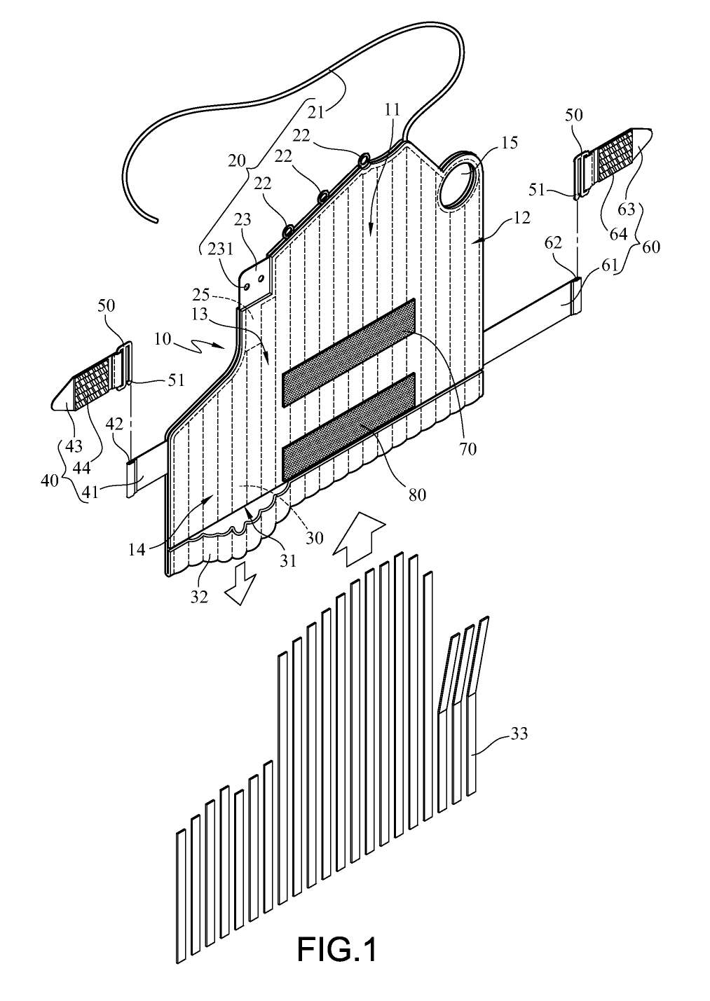

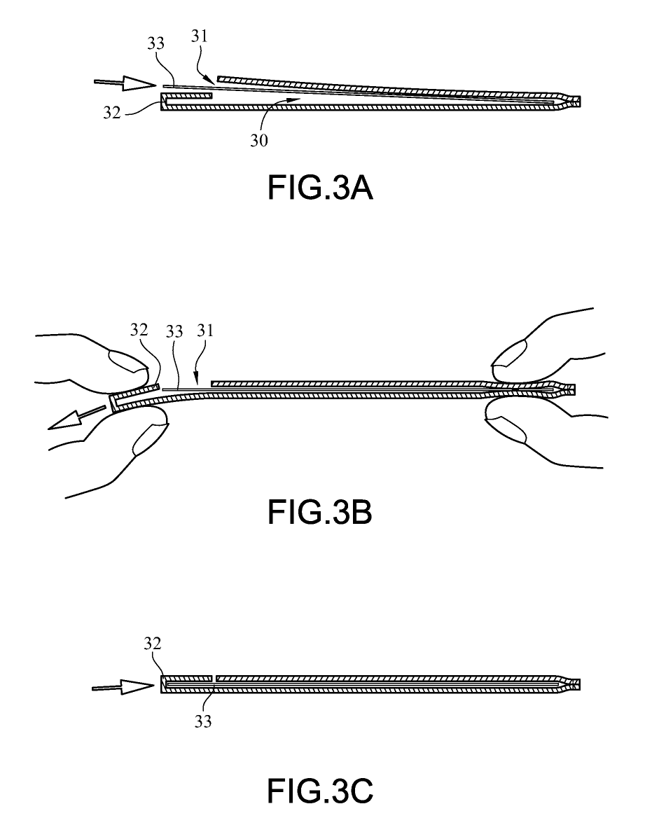

Referring to FIGS. 1 to 4C, a wrist support 1 provided in the present invention is used to wrap a hand back, a first metacarpal bone outer side of the hand, a fifth metacarpal bone outer side of the hand (the hand knife side), a wrist, and a portion of a forearm of a user, so as to protect and maintain the entire hand, the wrist, and the portion of the forearm in a fixed proper gesture and prevent an undesirable flexion when doing sports, which is further used as a correcting holder when the wrist is injured.

The wrist support 1 of the present invention includes a planar assembly 10, a finger-wearing set 20, at least one accommodating pocket 30, at least one supporting member 33, a first tightening strip set 40, and a second tightening strip set 60. The planar assembly 10 is formed by stitching two pieces of elastic materials with flexibility and pliability features (for example, materials such as Stretch Fabric or Neoprene). Therefore, the planar assembly 10 is configured as a planar plate. The planar assembly 10 includes a hand back portion 11, a thumb side portion 12, a hand knife portion 13, and a wrist portion 14, so as to completely wrap the hand back, the first metacarpal bone outer side of the hand, the fifth metacarpal bone outer side of the hand (the hand knife side), the wrist, and a portion of the arm of the user. The hand back portion 11 covers a portion of the hand back, an outer side of the wrist, and an outer side of a portion of the arm (i.e., a connecting line portion between an outer side portion of a second, third, fourth metacarpal bone and the outer side of the arm). The thumb side portion 12 covers the first metacarpal bone outer side of the hand, a radial side portion of the wrist, and a radial side portion of the arm (i.e., a connecting line portion between an outer side of a first metacarpal bone and an outer side of the radius). The hand knife portion 13 covers the fifth metacarpal bone outer side of the hand, an ulnar side portion of the wrist, and an ulnar side portion of the arm (i.e., a connecting line portion between an outer side of the fifth metacarpal bone and an outer side of the ulna). The wrist portion 14 covers an inner side of the wrist and an inner side of a portion of the arm. The planar assembly 10 is provided with a thumb fitting hole 15 for a thumb 91 of the user to penetrate there through. A finger-wearing set 20 is disposed on one side of the hand back portion 11 of the planar assembly 10 corresponding to the other fingers 92 of the user (i.e., at an edge position of a protruding top end of the hand back portion 11 of the planar assembly 10).

Referring to FIGS. 1 to 4C, the finger-wearing set 20 includes an elastic band 21, three rings 22, and a tab 23. The elastic band 21 is disposed at the edge of the protruding top end of the planar assembly 10 and located at a position between the hand back portion 11 and the thumb side portion 12. The tab 23 is disposed close to a protruding edge between the hand back portion 11 and the hand knife portion 13. The three rings 22 are disposed at a proper position of the protruding edge of the hand back portion 11. The elastic band 21 sequentially passes through the ring 22 and the tab 23, thereby forming at least one finger hole 24 for the other four fingers 92 except the thumb 91 to penetrate there through. The five fingers 91 and 92 are respectively fixed to the thumb fitting hole 15 and the finger hole 24, such that the planar assembly 10 is stably worn on the hand of the user, without sliding or displacing.

For the purpose of simplicity and being easily understood, in order to adjust an aperture size of the finger hole 24, two through-holes 231 are provided on the tab 23, which have diameters suitable for the elastic band 21 to pass there through. The tab 23 is made of a flat and rigid plastic material or metal material. The plastic material is suitable for being stitched with needles and fixed to the planar assembly 10. As for the metal material, needle holes need to be reserved thereon for stitching and fixing the metal material.

An unfixed end of the elastic band 21 sequentially passes through the ring 22, and then sequentially passes through the two through-holes 231 of the tab 23, and finally returns to and passes through a slit formed between the two through-holes 231 of the tab 23 where the elastic band 21 has passed through. Finally, the elastic band 21 is pulled tightly, so that the elastic band 21 is fixed and cannot slide. In this manner, at least one finger hole 24 is formed. The through-holes 231 may be round, rectangular, or of another shape applicable to a layout of the elastic band 21. Inner edges of the through-holes 231 are designed as tooth-shaped. A middle portion of a top edge of the tab 23 is also designed as tooth-shaped, such that it is difficult for the elastic band 21 to slide after being pulled tightly.

The two through-holes 231 of the tab 23 are used to adjust a length of the elastic band 21 and meanwhile enable the aperture size and tightness degree of each finger hole 24 to fit with size and adaptability of the user's fingers. With such a design, when the elastic band 21 needs to be adjusted, a portion of the elastic band 21 between the two through-holes 231 is pulled, so that the whole elastic band 21 can easily be loosened, so as to adjust its length once again.

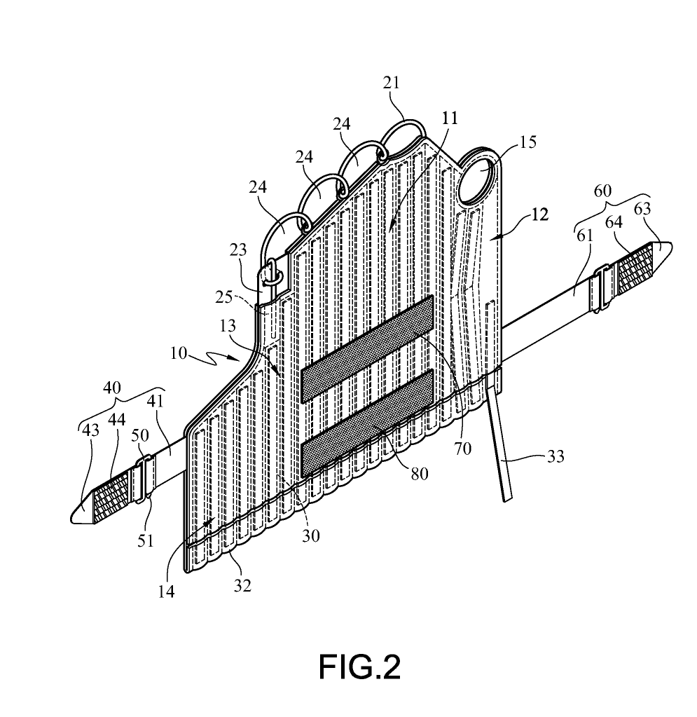

Referring to FIGS. 1 and 2, the planar assembly 10 of the present invention is further designed with a receiving pocket 25 for receiving a tail end of the elastic band 21 that drops down after the elastic band 21 passes through the tab 23, thereby improving the overall appearance of the wrist support 1. It should be noted that, the elastic band 21 in this embodiment passes through a plurality of ring 22. However, it should be understood by those skilled in the art that, on the proper position of the protruding edge of the hand back portion 11 of the planar assembly 10 may be directly opened with a plurality of holes for the elastic band 21 to pass there through, so as to form at least one finger hole 24, and it is not intended to be limited to the embodiments provided in the present invention.

Referring to FIGS. 1, 2, 4A, and 4B, the accommodating pockets 30 longitudinally correspond to a longitudinal connecting line between the hand, the wrist and the arm, and are evenly disposed in the hand back portion 11, the thumb side portion 12, the hand knife portion 13, and the wrist portion 14 of the planar assembly 10 in a parallel and adjacent manner. Each accommodating pocket 30 has an accommodating space therein and is respectively provided with an opening 31 and a cover 32 for covering the opening 31 on one side thereof corresponding to a linear edge of the planar assembly 10 (i.e., at the other end opposite to the finger-wearing set 20). The cover 32 is made of a material the same as the elastic material of the planar assembly 10, and stitched at a position of the planar assembly 10 corresponding to the opening 31. The supporting members 33 may be selectively mounted in the accommodating pockets 30 at different positions according to the user's actual requirements. In this embodiment, each of the accommodating pockets 30 is mounted with at least one supporting member 33 therein, such that the supporting members 33 of the wrist support 1 can completely wrap and fit tightly with the whole hand back, the first metacarpal bone outer side of the hand, the fifth metacarpal bone outer side of the hand (the hand knife side), the wrist, and a portion of the arm, so as to suppress incorrect flexion motions, thereby achieving an efficacy of protecting the hand, the wrist, and a portion of the arm at the same time.

The supporting members 33 may be made of a flat-rectangular or round-bar-shaped rigid elastic material. In this embodiment, the supporting members 33 are metal sheets having a bending property. However, it should be understood by those skilled in the art that, a material in different forms or different materials may also be selected as the supporting members 33, and it is not limited to the present invention. Referring to FIGS. 1 to 3C, widths of the supporting members 33 of the present invention are slightly smaller than sizes of the accommodating pockets 30. The accommodating pockets 30 are designed to have different lengths depending upon different portions. So the supporting members 33 have different length. The supporting members 33 are inserted into the accommodating pockets 30 via the openings 31. Closed ends of the accommodating pockets 30 (i.e., the other ends opposite to the openings 31) and the supporting members 33 are pressed with fingers. Since the whole planar assembly has desirable flexibility, the covers 32 are easily pulled outwards and deformed with respect to the planar assembly 10, so as to expose the openings 31. The force exerted to pull the covers 32 is not released until the supporting members 33 are disposed into the accommodating pockets 30, such that the accommodating pockets 30 completely wrap the supporting members 33. Since the supporting members 33 are completely accommodated in the accommodating pockets 30, they are difficult to be released out of the accommodating pockets 30 due to an external force or an excessive motion. In order to make the supporting members 33 of the thumb side portion 12 to fit with a connecting curve of the first metacarpal bone of the hand, the wrist, and the arm, the proper positions of the supporting members 33 at the thumb side portion 12 are moderately and slightly flexed, and the elastic stress thereof is maintained, so as to satisfy individual requirements of the user and achieve a desired comfort level. The supporting members 33 of the present invention are, for example, 0.5-1.0 mm thick and 5-10 mm wide metal sheets, all of the metal sheets are easily slightly bent, and the elasticity thereof is maintained.

Referring to FIGS. 1 to 4C, the first tightening strip set 40 of the present invention includes a first tightening strip 41 and a first Velcro portion 43. One end of the first tightening strip 41 is stitched to an upper end of an outer edge side of the wrist portion 14 of the planar assembly 10. The other end of the first tightening strip 41 is provided with a hook groove 42 formed by folding up and then stitching together. A hook structure 44 is disposed on a side surface of the first Velcro portion 43. The first Velcro portion 43 has an e-shaped hook ring 50. One end of the hook ring 50 is fixed on one end of the first Velcro portion 43, and the other end of the hook ring 50 is a hook portion 51. The hook portion 51 of the hook ring 50 is inserted into a slit of the hook groove 42 and fastened with the first tightening strip 41, which thus cannot be fallen off. In this way, the first tightening strip 41 and the first Velcro portion 43 form the first tightening strip set 40. Moreover, the first Velcro portion 43 and the first tightening strip 41 can be easily detached and separated or connected together. The second tightening strip set 60 includes a second tightening strip 61 and a second Velcro portion 63. One end of the second tightening strip 61 is stitched to a lower end of an outer edge side of the thumb side portion 12 of the planar assembly 10. The other end of the second tightening strip 61 is provided with a hook groove 62 formed by folding up and then stitching together. One side surface of the second Velcro portion 63 is provided with a hook structure 64. The second Velcro portion 63 has an e-shaped hook ring 50. One end of the hook ring 50 is fixed on one end of the second Velcro portion 63, and the other end of the hook ring 50 is a hook portion 51. The hook portion 51 of the hook ring 50 may be inserted into a slit of the hook groove 62 and fastened with the second tightening strip 61, which thus cannot be fallen off. In this way, the second tightening strip 61 and the second Velcro portion 63 form the second tightening strip set 60. Moreover, the second Velcro portion 63 and the second tightening strip 61 can be easily detached and separated or connected together.

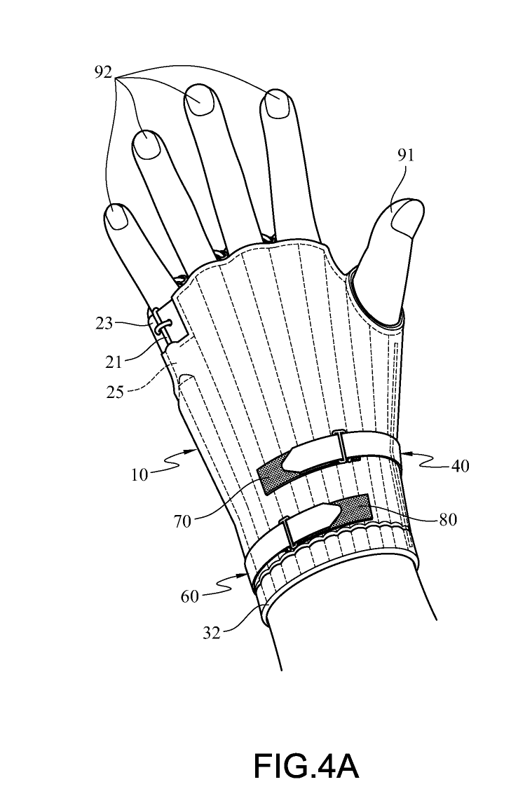

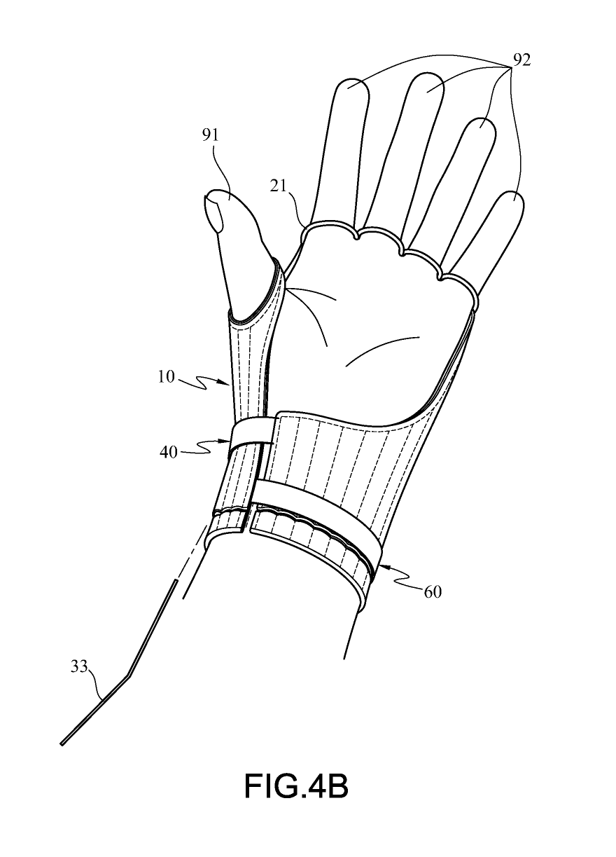

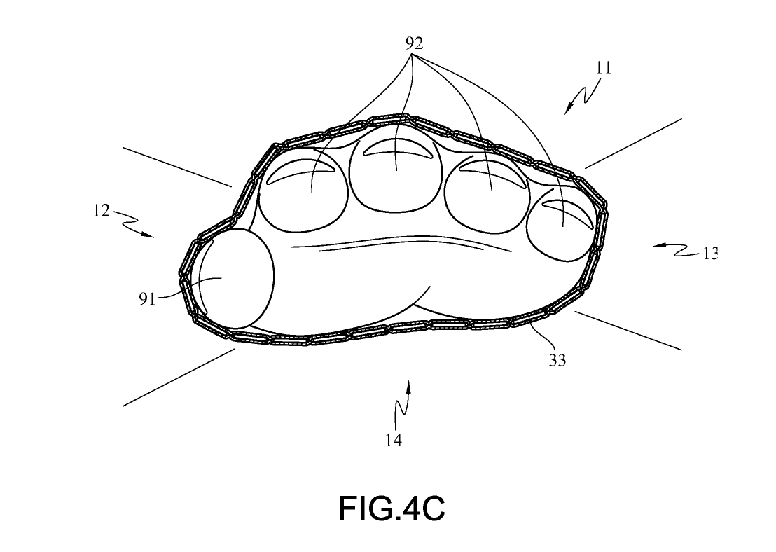

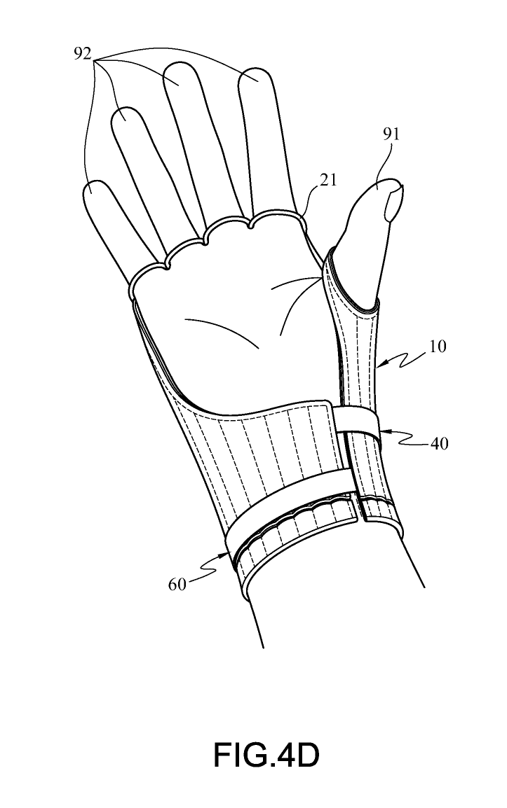

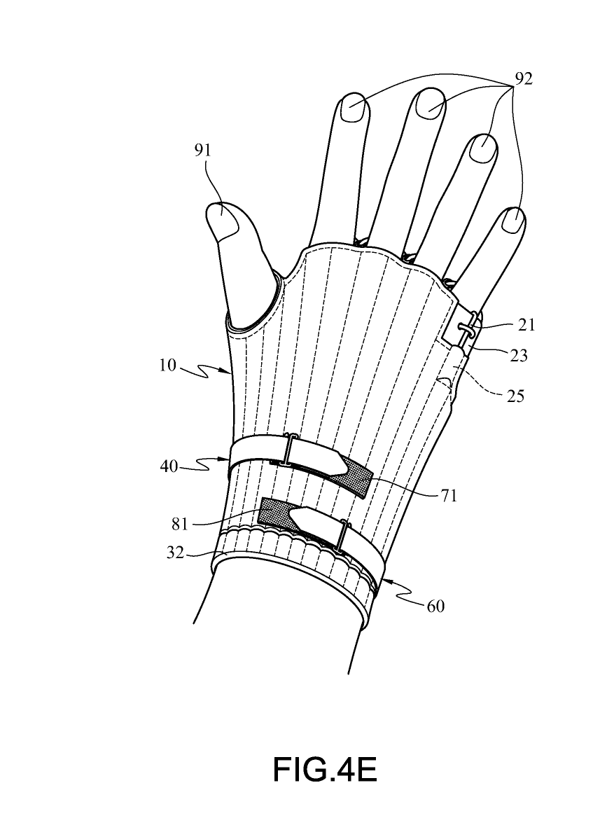

Referring to FIGS. 4A and 4E, adhering strips 70 and 71 having loop structures are respectively provided at the proper positions on the two surfaces of the hand back portion 11 of the planar assembly 10. The adhering strips 70 and 71 correspond to the first tightening strip set 40 in position and are aligned in line with the first tightening strip set 40. The adhering strips 70 and 71 are individually used to adhere with the hook structure 44 of the first Velcro portion 43 of the first tightening strip set 40. Adhering strips 80 and 81 having loop structures are provided in the same manner, which can be adhered with the second tightening strip set 60.

As shown in FIGS. 4A and 4E, before the wrist support 1 of the present invention is worn on the left hand of the user, the length of the elastic band 21 is adjusted first, so as to enable the aperture size, tightness, and intensity of the finger holes 24 to fit with the size and tightness feeling of the user's fingers 92. The thumb 91 passes through the thumb fitting hole 15 of the planar assembly 10, and the other fingers 92 sequentially pass through the finger holes 24 respectively. Then, the first tightening strip 41 of the first tightening strip set 40 is wrapped around the wrist, and the hook structure 44 of the first Velcro portion 43 is tightly adhered to the loop structure of the adhering strip 70 on a surface of the hand back portion 11. The second tightening strip 61 of the second tightening strip set 60 is fixed in a wrapping direction opposite to that of the first tightening strip 41, and the hook structure 64 of the second Velcro portion 63 is tightly adhered to the loop structure of the adhering strip 80 on a surface of the hand back portion 11. In this way, the wrist support 1 completely wraps the whole hand back, the first metacarpal bone outer side of the hand, the fifth metacarpal bone outer side of the hand, the wrist, and the portion of the forearm. Since the two tightening strip sets 40 and 60 are pulled tightly in different directions (left and right directions), the wrist support 1 is further stabilized, thereby finishing the wearing motion. If the Velcro portions 43 and 63 of the two tightening strip sets 40 and 60 are inversely arranged, the hook structures 44 and 64 thereof are tightly adhered to the adhering strips 71 and 81 on the other surface of the hand back portion 11 respectively. In this case, the wrist support 1 is inverted in direction and thus suitable for being worn on the right hand. If the outer surface of the planar assembly 10 adopts a material having a loop structure, the adhering strips 70, 71, 80, and 81 do not need to be disposed on the two surfaces of the planar assembly 10 any more.

In the present invention, the tightening strips 41 and 61 of the tightening strip sets 40 and 60 are made of resilient elastic bands, and each of the adhering strips 70, 71, 80, and 81 on the hand back portion 11 has a proper length. Therefore, the tightening strip sets 40 and 60 can be respectively fixed and tightened at a proper position on the arm close to the wrist, regardless of the arm size of the user.

Since the tightening and connecting effects of the thumb fitting hole 15, the finger holes 24, the first tightening strip set 40, and the second tightening strip set 60 suppress the two ends and middle portions of the supporting members 33 at the hand back portion 11, the supporting members 33 are tightly attached to a portion of hand back (i.e., positions of the second, third, and fourth metacarpal bones), the outer side of the wrist, and the outer side of the portion of the arm, and thus restrict the user's whole hand and wrist from easily flexing towards the hand back or flexing forward towards the palm center, thereby avoiding an excessive motion.

Since the tightening and connecting effects of the thumb fitting hole 15, the first tightening strip set 40, and the second tightening strip set 60 suppress the two ends and middle portions of the supporting members 33 at the thumb side portion 12, the supporting members 33 are tightly attached to the first metacarpal bone outer side of the hand, the radial side of the wrist, and the radial side of the arm (i.e., a connecting line portion between the outer side of the first metacarpal bone and the outer side of the radius), and thus restrict the user's whole hand and wrist from easily flexing towards the thumb 91 side, thereby avoiding an excessive motion. As shown in FIG. 1, proper portions in the middle of the supporting members 33 at the thumb side portion 12 corresponding to the position of the thumb 91 can be flexed to a certain extent, so as to cater to a connecting curve of the first metacarpal bone side of the palm and the arm.

Since the tightening and connecting effects of the finger holes 24, the first tightening strip set 40, and the second tightening strip set 60 suppress the two ends and middle portions of the supporting members 33 at the hand knife portion 13, the supporting members 33 at the hand knife portion 13 are tightly attached to the fifth metacarpal bone outside of the hand, the ulnar side portion of wrist, and a ulnar side portion of the arm (i.e., a connecting line portion between the outer side of the fifth metacarpal bone and the outer side of the ulna), and thus restrict the whole hand and the wrist from easily flexing towards the fifth metacarpal bone side and meanwhile further restrict the hand and the wrist from easily flexing towards the hand back or towards the palm center, thereby preventing an excessive motion. In addition, due to the tightening and connecting effects of the first tightening strip set 40 and the second tightening strip set 60, the supporting members 33 at the wrist portion 14 suppress and further protect an inner side of the wrist and an inner side of the arm. Since three directions of the hand back, the fifth metacarpal bone side of the hand (the hand knife s), and the first metacarpal bone side of the hand (the thumb side) can be suppressed simply by stably maintaining three portions of the hand, the wrist, and the arm, the highest degree of support is achieved.

The supporting members 33 of the wrist support 1 completely restrain the whole hand, the wrist, and a portion of the arm, which prevent them from excessively flexing and protect them from external impacts. Therefore, the wrist support 1 of the present invention is applicable to various sports that need to restrain the flexion of the wrist, such as golf, bowling, tennis, table tennis, or badminton.

Referring to FIGS. 1 and 2, since the first Velcro portion 43 and the second Velcro portion 63 of the present invention are easily detached from and assembled to the tightening strips 41 and 61, if the user intends to change a state of wearing the wrist support on the left hand as shown in FIG. 4A to a state of wearing it on the right hand as shown in FIG. 4E, hook portions 51 of the e-shaped hook rings 50 on the Velcro portions 43 and 63 of the two tightening strip sets 40 and 60 are taken out from the hook grooves 42 and 62 at one end of the tightening strips 41 and 61 respectively, and then the Velcro portions 43 and 63 are inverted by 180° and placed into the hook grooves 42 and 62 of the tightening strips 41 and 61 once again. Since the supporting members 33 of the thumb side portion 12 has a directionality feature, the supporting members 33 within the accommodating pockets 30 at the thumb side portion 12 need to be taken out, inverted, and then inserted into the original accommodating pockets 30 once again. If the supporting members 33 of the hand knife portion 13 are also in a slightly flexing configuration, they also need to be taken out, inverted, and then inserted into the accommodating pockets 30 once again. However, if the supporting members 33 are flat, no inverting motion is needed. In this manner, the wrist support 1 can be worn on the right hand of the user.

Moreover, as for the supporting members 33 at each portion, since different users have different strengths and different personal requirements on the suppressing force, additional supporting members 33 may be added to the accommodating pockets 30 at any portion that needs to enhance the suppression at any moment. If the user wants to reduce the suppressing force of the supporting members 33, the supporting members 33 may be easily taken out through the above manner for arranging the supporting members 33, that is, the supporting members 33 at any portion may be taken out at any time. The same effects can also be achieved by changing the type or thickness of the supporting members 33.

When playing golf, an ordinary golfer wears specialized golf gloves first and then wears the wrist support 1 of the present invention to correct the undesirable gestures, which has an excellent adaptability for suppressing the wrist and protecting the palm, the wrist, and the arm. The wrist support 1 of the present invention is also applicable to various other sports that need to suppress and protect the palm, the wrist, and the arm, such as bowling, baseball, table tennis, and tennis. A bowling player may directly wear the wrist support 1 of the present invention, and a baseball player may wear the wrist support 1 of the present invention first and then wear baseball gloves.

Besides the above sports, the wrist support 1 of the present invention can also be used as a correcting, fixing, and protecting device when the wrist is injured, so as to maintain the wrist in a fixed gesture and thus prevent the wrist from being injured again. After the wrist support 1 of the present invention is worn, the fingers still can move freely and flexibly, which promotes the blood circulation and enhances the rehabilitation effect. In addition, the number of the supporting members 33 can be increased or reduced at will, so as to satisfy different users' requirements. If the wrist in injured, after the wrist is maintained in a fixed gesture for a period of time, some of the supporting members 33 may be removed, so as to enable the wrist support 1 to be relatively flexible and allow the wrist to flex to a certain extent, thereby promoting the blood circulation and enhancing the rehabilitation effect. Alternatively, all the supporting members 33 may be removed, so that the wrist support 1 is used as a soft wrist brace.

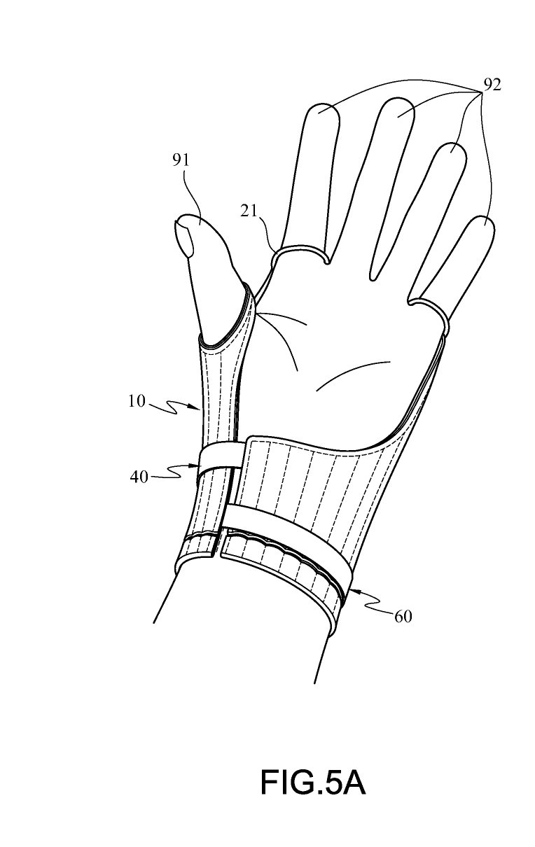

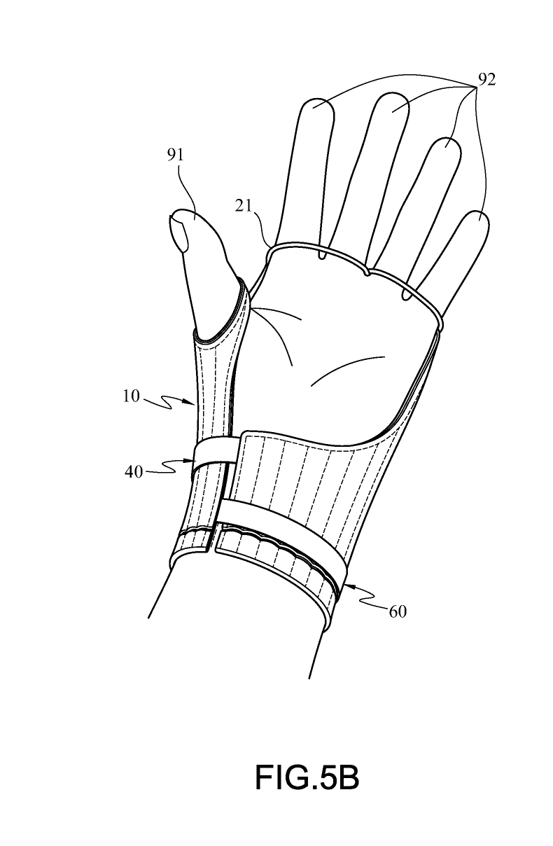

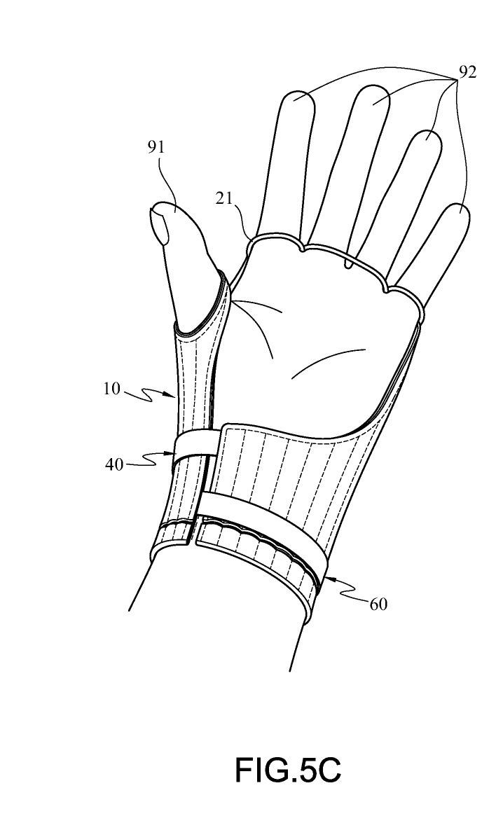

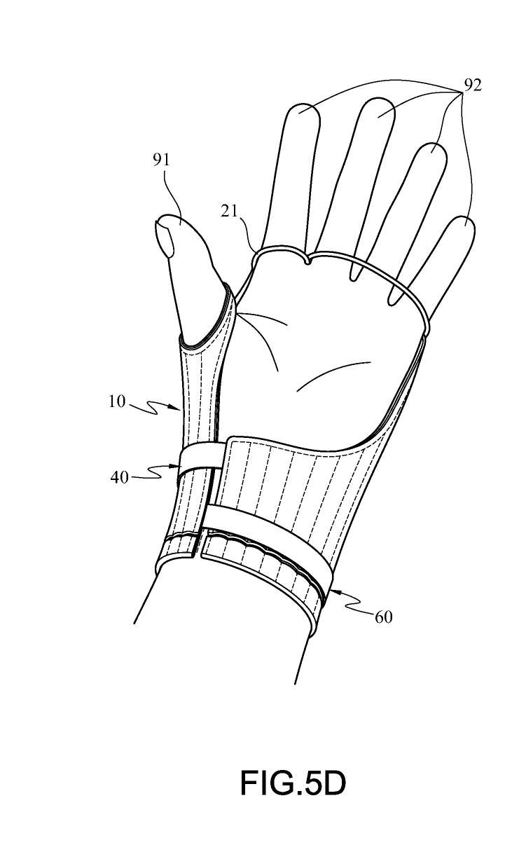

Referring to FIGS. 5A to 5E, besides the above manner that the finger-wearing set 20 is formed with four finger holes 24 for the user's four fingers 92 except the thumb 91 to pass there through respectively, the configuration for the elastic band 21 to pass through the ring 22 may also be adjusted and changed, such that the number and size of the finger holes 24 changes accordingly. After the thumb 91 passes through the thumb fitting hole 15, the user may selectively penetrate the fingers 92 into the finger hole 24 according to personal habits and comfort level. For example, the four fingers 92 are made to penetrate through one large finger hole 24 together. Alternatively, each two fingers 92 are made to penetrate through one finger hole 24. Alternatively, the index finger and the little finger are respectively made to penetrate through a single finger hole 24, and both the middle finger and the ring finger are made to penetrate through one finger hole 24. Alternatively, the index finger is made to penetrate through a single finger hole 24, and the other three fingers 92 are made to penetrate through one large finger hole 24 together. Alternatively, only the index finger is made to penetrate through a finger hole 24, and the other three fingers 92 are left alone, without penetrating through any finger hole. The above manners all can achieve the best effect and the highest level of comfort when wearing the wrist support 1.

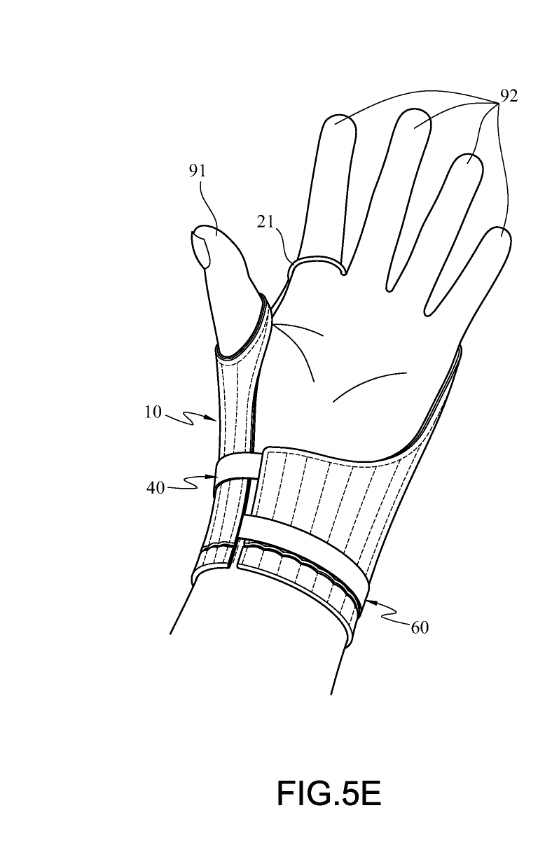

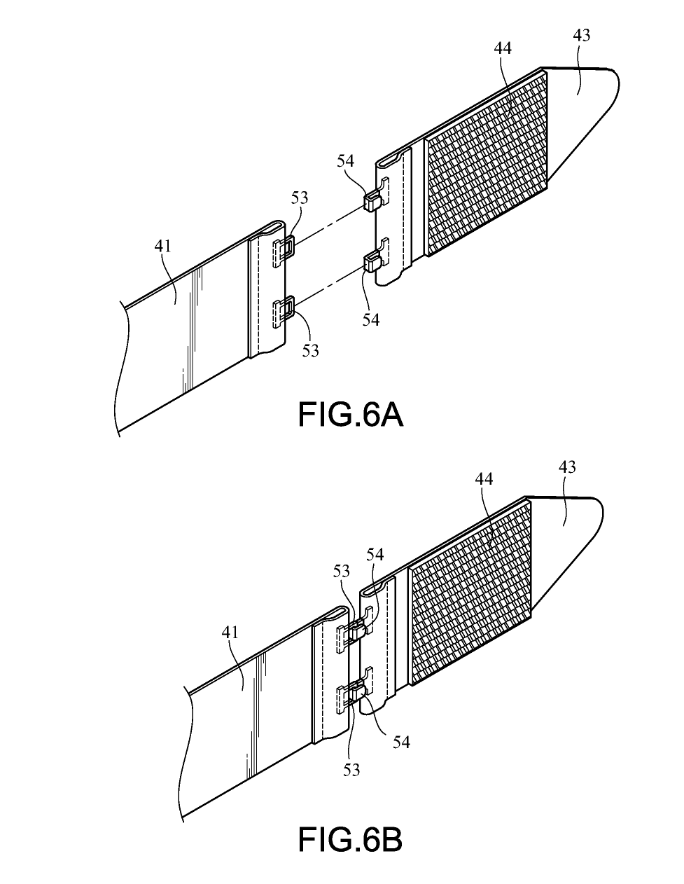

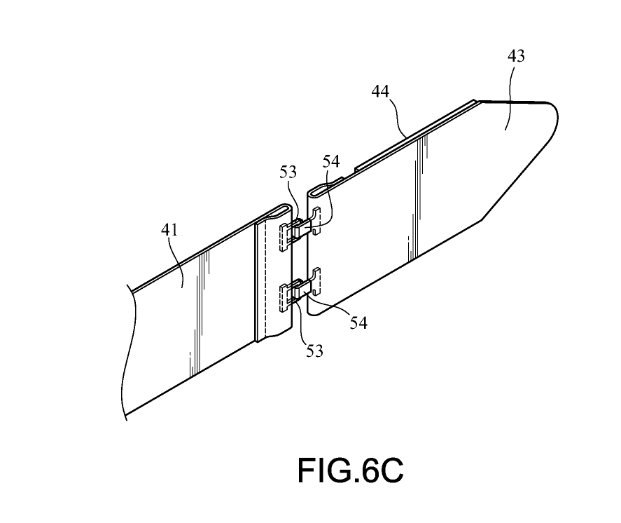

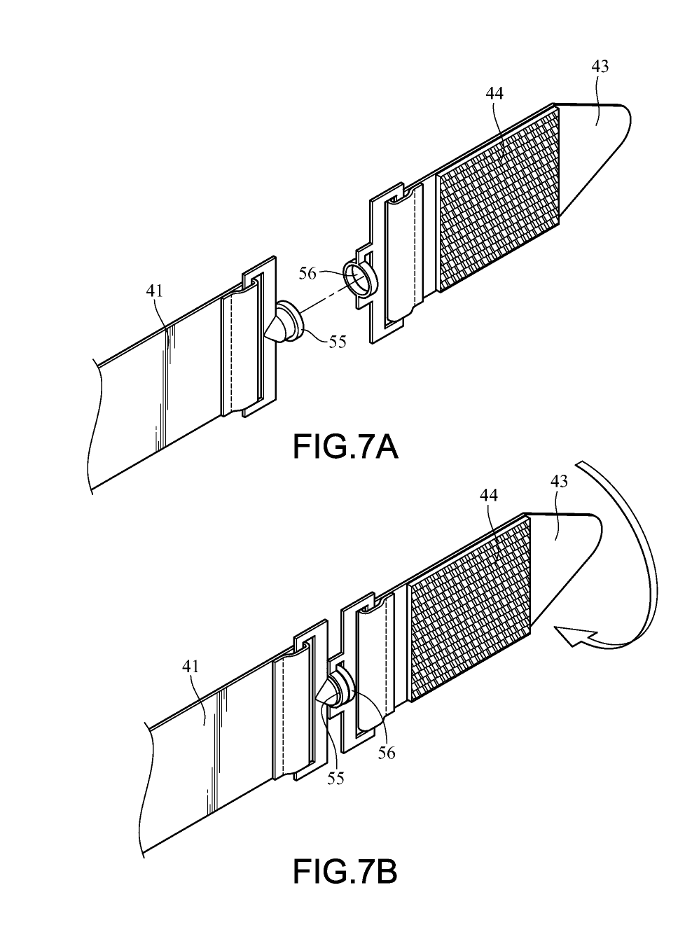

Referring to FIGS. 6A to 6C, besides the above connection manners, the tightening strips 41 and 61 and the Velcro portions 43 and 63 of the two tightening strip sets 40 and 60 of the present invention may also be designed in other forms. One end of each of the two tightening strips 41 and 61 is respectively provided with at least one snap ring 53. One end of each of the two Velcro portions 43 and 63 is respectively provided with at least one hook 54. The hooks 54 are fastened to the snap rings 53, such that the Velcro portions 43 and 63 can be easily detached from or assembled to the tightening strips 41 and 61. Alternatively, referring to FIGS. 7A and 7B, one end of each of the two tightening strips 41 and 61 is respectively provided with a sleeve shaft 55. One end of each of the two Velcro portions 43 and 63 is respectively provided with a shaft hole 56. The sleeve shafts 55 are movably inserted into the shaft holes 56, such that the Velcro portions 43 and 63 can be inversely rotated by 180° with respect to the tightening strips 41 and 61. However, it should be understood by those skilled in the art that, this is not limited to the embodiments provided in the present invention.

The wrist support 1 of the present invention has the following advantages.

1. The wrist support 1 of the present invention can protect the wrist and restrain the motion and flexion of the wrist, so as to maintain the wrist in a correct gesture when doing sports. For example, as for sports such as golf, bowling, table tennis, and tennis, incorrect flexing motion of the wrist can be adjusted in daily training or practice. When playing baseball, the wrist force can be enhanced if the wrist support 1 of the present invention is worn besides the baseball gloves. Alternatively, when grasping a table tennis racket, a tennis racket, or a badminton racket with a hand wearing the wrist support 1, the flexion extent of the wrist can be fixed, so that the player is cultivated into a habit with a desirable swinging motion.

2. The two Velcro portions 43, 63 can be detached from the two tightening strips 41, 61 and thus the disposing direction thereof can be changed, so the wrist support 1 is suitable for being worn on both the left hand and the right hand. The customers do not need to buy additional wrist braces for different hands. Therefore, the wrist support of the present invention can save the cost, and achieve the effects of simplicity, convenience, multifunction, and high economic benefits.

3. The supporting members 33 are placed into rectangular accommodating pockets 30 and completely wrapped by the covers 32, so as to prevent the supporting members 33 from falling out during usage. In addition, the number of the supporting members 33 can be easily increased or reduced, or the thickness or type of the supporting members 33 can be changed according to the user's personal habit and feeling. Moreover, since the wrist support 1 of the present invention is configured into an elastic structure, at least three supporting members 33 can be accommodated in one accommodating pocket 30.

4. When the wrist support 1 is worn, the user's thumb passes through the thumb fitting hole 15, so as to ensure that the wearing position of the wrist support 1 is right aligned with the direction of the palm, the wrist, and the forearm. The other fingers are made to pass through the finger holes 24, which can assist to further fix the wearing position of the wrist support 1, thereby preventing the wrist support 1 from sliding easily. Therefore, the longitudinal direction of the supporting members 33 stably corresponds to a longitudinal connecting line between the metacarpal bones of the palm and the arm. Furthermore, under the connecting and fixing effects of the first tightening strip set 40 and the second tightening strip set 60, the supporting members 33 are completely attached to the hand back, the first metacarpal bone outer side of the hand, the fifth metacarpal bone outer side of the hand (the hand knife side), the wrist, and a portion of the forearm tightly.

5. The size of the finger holes 24 formed by elastic bands can be adjusted to fit with the size and tightness feeling of the user's fingers. Moreover, the finger-wearing set 20 can be adjusted to different combinations of finger holes 24 according to user's personal requirements, so as to satisfy different users' wearing requirements and feelings.

6. When the wrist is injured, the wrist support 1 of the present invention can be used as a protector for holding the wrist joint completely, thereby preventing the wrist from being injured once again. In this case, the wrist support of the present invention achieves functions and benefits of a rigid wrist brace.

7. If the wrist is injured, after the wrist support 1 is worn as a rigid wrist brace for a period of time, the number of the used supporting members 33 may be gradually reduced or even all the supporting members 33 may be removed, so as to enable the wrist to flex moderately, thereby promoting the blood circulation and enhancing the rehabilitation effect. In this case, the wrist support 1 of the present invention achieves benefits of a soft wrist brace.

The above designs conform to ergonomics requirements and can offer the highest level of comfort to the user who wears the wrist support of the present invention.

What is claimed is:

1. A wrist support, suitable for wrapping a hand, a wrist, and a portion of an arm of a user, comprising:
a planar assembly, comprising a hand back portion, a thumb side portion, a hand knife portion, and a wrist portion, for wrapping a hand back, a first metacarpal bone outer side of the hand, a fifth metacarpal bone outer side of the hand, the wrist, and the portion of the arm, wherein a thumb fitting hole is provided at a position of the planar assembly corresponding to the thumb of the hand for the thumb of the hand to penetrate there through;
a finger-wearing set, disposed on one side of the hand back portion of the planar assembly corresponding to the other fingers except the thumb of the hand, wherein the finger-wearing set is provided with at least one finger hole for the other fingers except the thumb of the hand to penetrate there through;
at least one accommodating pocket, disposed on the planar assembly, wherein the accommodating pocket has an accommodating space therein and is provided with an opening and a cover for covering the opening on the other end thereof opposite to the finger-wearing set;
at least one supporting member, for being selectively accommodated in the accommodating pocket;
a first tightening strip set, comprising a first tightening strip and a first Velcro portion, wherein one end of the first tightening strip is fixed to an outer edge side of the wrist portion of the planar assembly, and the first Velcro portion is detachably mounted on the other end of the first tightening strip and adhered to the hand back portion; and
a second tightening strip set, comprising a second tightening strip and a second Velcro portion, wherein one end of the second tightening strip is fixed to an outer edge side of the thumb side portion of the planar assembly, and the second Velcro portion is detachably mounted on the other end of the second tightening strip and adhered to the hand back portion.

2. The wrist support according to claim 1, wherein the finger-wearing set comprises an elastic band, at least one ring, and a tab; one end of the elastic band is disposed at a protruding edge position between the hand back portion and the thumb side portion of the planar assembly; the ring is disposed on one side of the planar assembly corresponding to the other fingers except the thumb of the hand; the tab is disposed close to a protruding edge position between the hand back portion and the hand knife portion of the planar assembly; the tab is provided with at least two through-holes; and the elastic band passes through the ring and the through-holes of the tab and is then fixed, thereby forming the finger hole.

3. The wrist support according to claim 2, wherein inner edges of the through-holes are configured into tooth-shaped.

4. The wrist support according to claim 1, wherein two surfaces of the hand back portion of the planar assembly are respectively provided with two adhering strips, and each of the adhering strips has a loop structure, one surface of the first Velcro portion and one surface of the second Velcro portion are respectively provided with a hook structure, and the loop structure is respectively bonded to the hook structure, such that the first Velcro portion and the second Velcro portion are respectively adhered to the two adhering strips on one surface of the hand back portion.

5. The wrist support according to claim 1, wherein the other end of the first tightening strip and the other end of the second tightening strip are respectively provided with a hook groove; one end of the first Velcro portion and one end of the second Velcro portion are respectively provided with a hook ring; the hook ring has a hook portion; and the hook portion of the hook ring is inserted into the hook groove and respectively fastened to each of the two tightening strips, such that the two Velcro portions are respectively detachably mounted on the two tightening strips.

6. The wrist support according to claim 1, wherein the other end of the first tightening strip and the other end of the second tightening strip are respectively provided with at least one snap ring; one end of the first Velcro portion and one end of the second Velcro portion are respectively provided with at least one hook; and the hook is fastened to the snap ring, such that the two Velcro portions are respectively detachably mounted on the two tightening strips.

7. The wrist support according to claim 1, wherein the other end of the first tightening strip and the other end of the second tightening strip are respectively provided with a sleeve shaft; one end of the first Velcro portion and one end of the second Velcro portion are respectively provided with a shaft hole; and the sleeve shaft is movably inserted into the shaft hole, such that the two Velcro portions are rotated by 180° with respect to the two tightening strips.

8. The wrist support according to claim 1, wherein the planar assembly is further provided with a receiving pocket for receiving a tail end of the elastic band.

9. The wrist support according to claim 1, wherein the cove is deformed with respect to the planar assembly to expose the opening, such that the supporting member is mounted in the accommodating pocket from the opening.

10. The wrist support according to claim 1, wherein the planar assembly is made of an elastic material.

11. The wrist support according to claim 1, wherein the first tightening strip and the second tightening strip are made of an elastic material.
